(19) United States
(12) Patent Application Publication
(10) Pub. No.: US 2025/0127544 A1
(43) Pub. Date: Apr. 24, 2025

(54) SYSTEM AND DEVICE FOR PERFORMING VERTEBRAL AUGMENTATION

(71) Applicant: EncapsuleMED, Inc., Newark, DE (US)

(72) Inventors: Abdulrazzaq ALOBAID, Safat (KW); Hunter MULLINS, Lexington, KY (US)

(73) Assignee: EncapsuleMED, Inc., Newark, DE (US)

(21) Appl. No.: 18/705,966

(22) PCT Filed: Oct. 27, 2022
(86) PCT No.: PCT/US2022/048107

§ 371 Date: Apr. 29, 2024

Related U.S. Application Data
(60) Provisional application No. 63/272,523, filed on Oct. 27, 2021.

(51) Int. Cl. A61B 17/70; A61B 17/00; A61B 17/88; A61B 90/00

(52) U.S. Cl. CPC A61B 17/7097; A61B 17/8833; A61B 2017/00004; A61B 2017/00557; A61B 2090/3966

(57) ABSTRACT

A system for performing a vertebral augmentation procedure is provided. The system may comprise a delivery device and an encapsulation balloon. The delivery device may include an inflation tube, a push tube provided over a proximal portion of the inflation tube, and a push handle. The inflation tube may have a delivery cannula and an injection port, wherein bone cement can flow through the delivery cannula and out of the injection port. The encapsulation balloon is configured for receiving cement and may be provided over a distal portion of the inflation tube such that the port is provided within the encapsulation balloon.

### CROSS REFERENCE TO RELATED APPLICATION

The present application claims the benefit of to U.S. Provisional Application No. 63/272,523, filed on 27 Oct. 2021, the entire contents of which are incorporated herein by reference. A claim of priority to all, to the extent appropriate, is made.

### FIELD OF THE INVENTION

The present disclosure relates to novel and advantageous devices and systems for performing vertebral augmentation. Particularly, the present disclosure relates to novel and advantageous balloons and delivery systems for performing vertebral augmentation using a balloon. More particularly, the present disclosure relates to a encapsulation balloon and delivery catheter for inserting the balloon in vivo and filling the balloon with cement.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Vertebral compression fractures (VCFs) occur when a vertebral body in the spine collapses, which can lead to severe pain, deformity, and loss of height. These fractures commonly occur in the thoracic spine, especially in the lower thoracic spine.

Vertebroplasty is a common treatment for VCFs and was introduced in the United States in the early 1990s. Vertebroplasty is a minimally invasive procedure where high pressure cement is injected into the vertebra. A small needle containing bone cement is injected into the collapsed vertebra. The cement hardens within minutes, strengthening and stabilizing the fractured vertebra. Complications can arise with cement leakage. These may comprise, for example, damage to neural structures, lungs, or leakage into vascular systems.

Since the introduction of vertebroplasty, vertebroplasty procedures expanded as one of the treatment modalities to treat pathologic fractures including vertebral body osteoporotic fractures. The thermal effect and the vertebral body augmentation helped with pain control of these cases. The procedure is done utilizing image guided open or percutaneous techniques where PMMA is mixed with radio-opaque material and then injected into the vertebral body through pedicle portals. It can be done under either local or general anesthesia. The cement is forcibly injected through syringes attached to the portals. This is a high-pressure system and has led in many cases to cement leak from the vertebral body. The reported rate of cement extravasation was 76% when computerized tomography (CT) scans were used. If leakage happens in the neural canal, it can cause neurologic deficits. Other complications reported include pulmonary embolism, infection, and incomplete stabilization. Kyphoplasty was later introduced to minimize risks of cement leak and achieve better sagittal alignment.

Kyphoplasty, a balloon-assisted vertebroplasty alternative for treatment of VCFs, is a modification of the vertebroplasty technique and is a method of vertebral augmentation. Kyphoplasty is a contemporary balloon-assisted vertebroplasty alternative for treatment of vertebral body compression fractures. Kyphoplasty utilizes low pressure cement injection to decrease the problem of cement leakage. Kyphoplasty involves injection of bone cement into a mechanically created bone void within a vertebral body.

More specifically, in kyphoplasty, a balloon is used to create a void in the vertebral space. Kyphoplasty modified the vertebroplasty technique by introducing an inflatable balloon before the cement is injected. The balloon serves to elevate the end plates in an attempt to restore the vertebral body height and sagittal alignment by correcting the kyphosis. The balloon is inserted into the structurally compromised vertebral body, often through a cannula, The balloon is then inflated under high pressure. It is thought that the expanding balloon disrupts the cancellous bone architecture and physiological matrix circumferentially and directs the attendant bony debris and physiological matrix toward the inner cortex of the vertebral body vault, i.e. restores the height of the vertebra. The balloon is then deflated and removed, leaving a bony void or cavity. The remaining void or cavity is filled with an appropriate biomaterial, most often bone cement such as polymethylmethacrylate (PMMA). In most cases, the treatment goals are to reduce or eliminate pain and the risk of progressive fracture of the vertebral body and its likely resulting morbidity, complications, and disability.

An advantage of kyphoplasty is creating a void after balloon removal. This allows cement to be injected in a low pressure void system compared to the high pressure vertebroplasty systems.

A common risk of kyphoplasty is leakage of the PMMA from the cavity, which may cause nerve injury, infection, numbness, or spinal cord compression, or may require corrective procedures resulting from leakage of the cement.

Thus, an encapsulation balloon for use in vertebral augmentation solving the aforementioned problems is desired.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a system for performing a vertebral augmentation procedure including a delivery device and an encapsulation balloon. The delivery device may include an inflation tube, a push tube provided over a proximal portion of the inflation tube, and a push handle. The inflation tube may have a delivery cannula and an injection port, wherein bone cement can flow through the delivery cannula and out of the injection port. The encapsulation balloon is configured for receiving cement and may be provided over a distal portion of the inflation tube such that the port is provided within the encapsulation balloon.

The present disclosure, in one or more embodiments, additionally relates to a system for performing a vertebral augmentation procedure including a delivery catheter and an encapsulation balloon. The delivery catheter may comprise a central tube, a distal tip assembly, a dispensing tip, and a handle. The central tube includes a proximal end and a distal end and the distal tip assembly is provided at the distal end. The distal tip assembly includes a port wherein bone cement can flow through the central tube, to the distal tip assembly, and out of the port. The dispensing tip is configured for receiving a cement cannula. The handle is removably coupled to the central tube and is configured to receive the dispensing tip. The encapsulation balloon is provided over the distal tip assembly and is configured for receiving bone cement.

The present disclosure, in one or more embodiments, further relates to a system for performing a vertebral augmentation procedure comprising a cement encapsulation system, a catheter delivery system, and an encapsulation balloon. The cement encapsulation system may comprise a cement reservoir having a proximal end and a reservoir distal tip, a stopcock, a syringe for removing air, and a tube. The delivery catheter system may comprise a delivery catheter and a handle. The delivery catheter may comprise a central tube having a distal end and a proximal end, a catheter distal tip assembly. The distal tip assembly may include a port, wherein bone cement can flow through the central tube, to the distal tip assembly, and out of the port. The handle may be removably coupled to the central tube. The cement reservoir may be removably coupled to the delivery catheter. The encapsulation balloon may be provided over the catheter distal tip assembly and may be configured for receiving bone cement

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1a illustrates a method of performing vertebral augmentation using a balloon, in accordance with one embodiment.

FIG. 1b illustrates a system for performing vertebral augmentation using a balloon, in accordance with one embodiment.

FIG. 2a illustrates a delivery catheter, without balloon, in accordance with one embodiment.

FIG. 2b illustrates the delivery catheter of FIG. 2a with a balloon provided over a distal end of the delivery catheter for insertion into the target area, such as a vertebral body.

FIG. 3a illustrates an access catheter for accessing a vertebral body, in accordance with one embodiment.

FIG. 3b illustrates a delivery catheter in accordance with one embodiment.

FIG. 4 illustrates a close up view of an aspects of a distal portion of the delivery catheter, in accordance with one embodiment.

FIG. 5a illustrates a close up view of an aspects of a distal portion of the delivery catheter, in accordance with one embodiment.

FIG. 5b illustrates a close up view of an aspects of a distal portion of the delivery catheter, in accordance with one embodiment.

FIG. 6 illustrates a close up cross sectional view a distal portion of the delivery catheter with a distal tip assembly provided thereon, in accordance with one embodiment.

FIG. 7 illustrates a close up cross sectional view a distal portion of the delivery catheter with a distal tip assembly provided thereon, in accordance with one embodiment.

FIG. 8 illustrates a close up cross sectional view a distal portion of the delivery catheter with a distal tip assembly provided thereon, in accordance with one embodiment.

FIG. 9a illustrates an aspect of a connector piece, in accordance with one embodiment.

FIG. 9b illustrates an aspect of a connector piece, in accordance with one embodiment.

FIG. 10 illustrates a handle for a delivery catheter, in accordance with one embodiment.

FIG. 11 illustrates a handle for a delivery catheter, in accordance with one embodiment.

FIG. 12 illustrates the delivery catheter with a biodegradable balloon inflated at the distal end, in accordance with one embodiment.

FIG. 13a illustrate a cement cannula, in accordance with one embodiment.

FIG. 13b illustrate a cement cannula, in accordance with one embodiment.

FIG. 13c illustrate a cement cannula, in accordance with one embodiment.

FIG. 14a illustrates an encapsulation balloon, in accordance with one embodiment.

FIG. 14b illustrates an encapsulation balloon, in accordance with one embodiment.

FIG. 14c illustrates an encapsulation balloon, in accordance with one embodiment.

FIG. 15a illustrates inflation of an encapsulation balloon, in accordance with one embodiment.

FIG. 15b illustrates deployment of an encapsulation balloon, in accordance with one embodiment.

FIG. 16a illustrates a cement encapsulation catheter as removed from packaging, in accordance with one embodiment.

FIG. 16b illustrates a balloon protective sheath removed from the cement encapsulation catheter, in accordance with one embodiment.

FIG. 16c illustrates a stiffening wire handle provided inserted in the cement encapsulation catheter, in accordance with one embodiment.

FIG. 17 illustrates a bone cement mixer coupled to a cement reservoir, in accordance with one embodiment.

FIG. 18a illustrates a cement encapsulation catheter with a stylet inserted therein, in accordance with one embodiment.

FIG. 18b illustrates the cement encapsulation catheter of FIG. 18a with the stylet removed therefrom, in accordance with one embodiment.

FIG. 19a illustrates introduction of a delivery catheter to the vertebral space, in accordance with one embodiment.

FIG. 19b illustrates deployment of a cement encapsulation balloon through a delivery catheter, in accordance with one embodiment.

FIG. 19c illustrates inflation of a cement encapsulation balloon, in accordance with one embodiment.

### DETAILED DESCRIPTION

The present disclosure relates to a novel and advantageous system and device for treatment of vertebral compression fractures using vertebral augmentation such as vertebroplasty or kyphoplasty. Such system and device may be used to encapsulate cement in a vertebral augmentation procedure. In particular, the present disclosure relates to an encapsulation balloon and delivery catheter for inserting the balloon in vivo and filling the encapsulation balloon with cement.

The system described herein comprises a catheter system for safe delivery of cement into the vertebral body following bone dilation and a method of making such system. The system may use an absorbable encapsulation balloon that utilizes a biodegradable matrix. The balloon may comprise an inflatable portion and an interior shaft. The encapsulation balloon is inserted in similar fashion to a balloon used in standard kyphoplasty but is not retrieved. The encapsulation balloon is maintained in situ and later filled with PMMA or other bone cement material. The balloon may be sealed with a valve that substantially prevents cement extravasation, thus minimizing complications related to cement leak. The encapsulation balloon may be configured to dissolve at a later stage after the PMMA is solid.

In one embodiment, an encapsulation balloon for vertebral augmentation procedures, such as vertebroplasty or kyphoplasty, is provided. The encapsulation balloon may be a biodegradable balloon that leaves hardened cement in place after degradation or may be a permanent balloon that remains in place with the hardened cement. In a biodegradable, or bio-absorbable, encapsulation balloon embodiment, the encapsulation balloon may comprise a biocompatible, biodegradable synthetic material. For example, the balloon may comprise a resomer and dicholoromethane mix. In one embodiment, the balloon comprises a Resomer LC 703 S material. The biodegradable material, or Resomer, may be mixed with a solvent such as Sigma-Aldrich PN 34856-4L Dichloromethane (DCM). The mix ratio may be, for example, 1.6 grams Resomer with 5 cc of Dichlorometane. In other embodiments, the balloon may comprise a PLA, PLGA, pol-caprolactone, polydiaxone, poly(lactide-co-epsilon-caprolactone), combinations thereof, or other biodegradable and/or bioabsorbable material that dissolves after the cement material within the encapsulation balloon solidifies. It is to be understood that any suitable type of biodegradable material may be utilized, including for example, materials having polyglycolic acid (PGA), polylactic acid (PLA), and/or copolymers thereof. In some embodiments, a bio-absorbable encapsulation balloon comprising a bio-absorbable fibrous mesh may be provided.

The balloon may comprise an inflatable portion and an interior shaft within the inflatable portion. In general, the balloon may be formed by forming an inflatable portion, forming an interior shaft, and assembling the balloon by providing the interior shaft within the inflatable portion.

In a non-biodegradable encapsulation balloon embodiment, the cement balloon is not biodegradable and may exist in the body and may comprise, for example, silicone material.

Throughout this document, like element numbers may be used to identify like components in all drawings. In some instances, a component may not be specifically described with reference to a figure and description given elsewhere may apply.

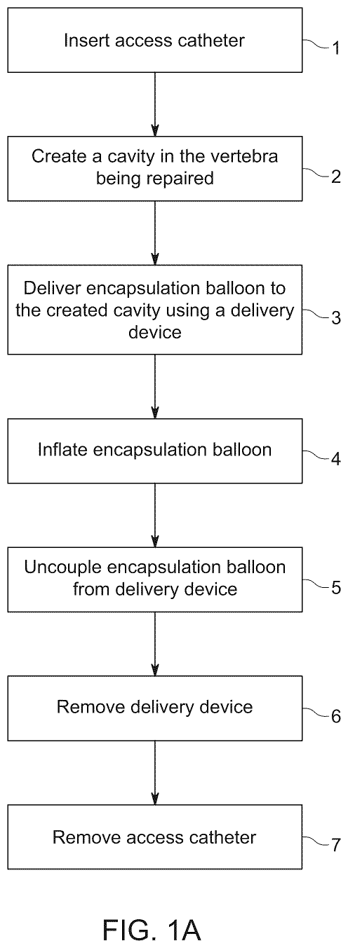

FIG. 1a illustrates a method of performing vertebral augmentation. As a first step, an access catheter is inserted through an incision in the back to access the vertebral body, shown at step 1. More specifically, the access catheter is pushed along a path through the pedicle of the vertebral body and into the fractured area.

A cavity is created inside the vertebra to be prepared, shown at step 2. This can be done in any suitable manner. For example, this may be done using a balloon tamp (or kyphoplasty balloon). The balloon tamp is inserted through the access catheter and into the vertebral body. The balloon of the balloon tamp is inflated and compacts the soft inner bone, thereby creating a cavity inside the vertebra, and returning the vertebral body to a natural height. The balloon tamp may then be deflated, and removed. In some embodiments, this step may be combined with, and done by, inflation of the encapsulation balloon, described below.

An encapsulation balloon is delivered to the created cavity, shown at step 3. This may be done by coupling the encapsulation balloon to a delivery device such as a delivery catheter. More specifically, the encapsulation balloon may be coupled to an inflation cannula or tube. The encapsulation balloon and inflation cannula are inserted through the access catheter to access the cavity. Insertion may be done using radiographic imaging to position the device.

The encapsulation balloon is inflated to generally fill the cavity, shown at step 4. Inflation is done by inserting bone cement, or other suitable material, into the encapsulation balloon. This may be done using the inflation cannula. When the desired quantity of cement has been injected into the encapsulation balloon, the encapsulation balloon is uncoupled from the delivery device, shown at step 5, and the delivery device is removed, shown at step 6. The access catheter may be removed, shown at step 7.

In some embodiments, a minimal volume, for example 2 ml, of cement may be deployed into the vertebral space prior to filling of the encapsulation balloon, or after filling of the encapsulation balloon but before the encapsulated cement is solidified, to facilitate fixation of the encapsulation balloon, to assist in pain relief, and/or to maintain vertebral stability. The balloon may be designed to allow connection between outside cement and cement inside the balloon. More specifically, the interdigitation of the cement within the trabecula may contribute to immediate pain relief and maintenance of vertebral stability. In one embodiment, the balloon facilitates connection of cement outside of the balloon and cement inside the balloon.

Accordingly, a system for performing vertebral augmentation procedures is provided. In one embodiment, the system includes a delivery device (also referred to as a delivery catheter), a balloon tamp (also referred to as a kyphoplasty balloon), and an encapsulation balloon. The balloon tamp may be used for creating a void in the vertebra and the encapsulation balloon may be used for filling the void created. In some embodiments, the encapsulation balloon may be used for both creating a void in the vertebra and for filling the created void. In such embodiment, no separate balloon tamp is provided.

The system may further comprise an access catheter and a cement cannula. The access catheter may be used to access the vertebral body and may have a tool, such as a stylet, centrally located therein to assist in forming the path to the vertebral body. In some embodiments, a catheter with a balloon tamp may be inserted through the access catheter, the balloon of the balloon tamp inflated to create a void in the vertebral body, the balloon deflated, and the balloon tamp and catheter removed from the access catheter. An encapsulation balloon may be attached to a delivery catheter and the delivery catheter inserted into the access catheter to position the encapsulation balloon in the void in the vertebral body. The cement cannula may engage the delivery catheter to deliver cement to the encapsulation balloon through the delivery catheter. The cement cannula and the delivery catheter may be removed from the access catheter and the access catheter may be removed from the body, all while leaving the encapsulation balloon and cement therein in situ.

While the term “cement” is used herein, it is to be appreciated that any suitable bone replacement material may be used. As is known to those skilled in the art, bone replacement material may be biological, such as demineralized bone matrix, platelet-rich plasma, hydroxyapatite, adjunction of growth factors (like bone morphogenetic protein), or synthetic, such as calcium sulfate, tri-calcium phosphate ceramics, bioactive glasses, or polymer-based substitutes. In general, injectable polymethyl methacrylate (PMMA), composite bone cement, biodegradable bone cement, calcium phosphate cement (CPC), calcium sulfate cements, composite resin materials, and the like may be used. In a specific embodiment, G21 V-Fast/V-Steady™ Bone Cement may be used with the cement encapsulation system disclosed herein. No preference is given to any particular bone replacement material herein and any suitable bone replacement material may be used with the system disclosed herein. In some embodiments a low viscosity cement may be used.

FIG. 1b illustrates a system for performing vertebral augmentation, in accordance with one embodiment. The system for performing vertebral augmentation may include a cement encapsulation system and a catheter delivery system. In the embodiment shown, the vertebral augmentation system includes a delivery catheter 200, a cement encapsulation catheter 202 (and associated system), a 3-way stopcock or valve 204, a VacLok syringe 206 (or similar device), a tube 208, and a pump 210 (optionally hand operated).

The cement encapsulation system may comprise a cement reservoir (barrel) 212, a hydraulic pump 210, such as a hand-operated hydraulic pump, that may be provided pre-filled with sterile water, and a flexible tube 208 connecting the reservoir to the pump. The reservoir 212 is connected to a catheter delivery system which itself comprises a delivery catheter 200. System accessory instruments may include mixing tools such as a mixer with cement reservoir connection, a VacLok syringe 206, and a 3-way stopcock 204. The VacLok syringe 206 and 3-way stopcock 204 facilitate air removal from the catheter delivery system prior to cement injection. The cement encapsulation system may be used with an introducer cannula having, for example, stainless steel tubing. In some embodiments, the stainless steel tubing may be 5 inches (150 mm) or less, and have an inner diameter of 8 G or more.

The catheter delivery system, described more fully below, may comprise a cannula, a push tube, and a stylet. The catheter delivery system facilitates percutaneous access and delivery of the encapsulation into the vertebral body and cement to the encapsulation balloon. The size of cannula used may be selected based on a patient's anatomy and pathology.

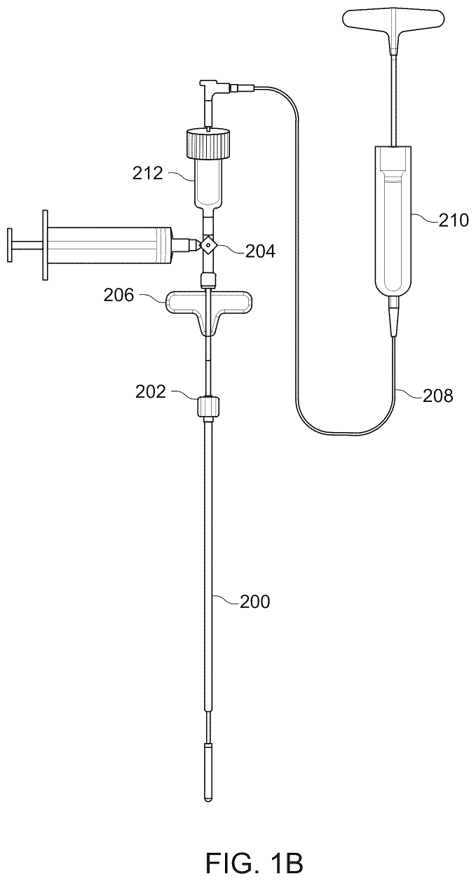

FIG. 2a illustrates a delivery catheter or a catheter delivery system, in accordance with one embodiment. In FIG. 2a, the delivery catheter is shown without an encapsulation balloon. In the embodiment shown, the delivery catheter 100 includes an inflation tube 102, a push tube 104, a push handle 106, and an inflation luer 108. The inflation tube 102 may be skived or otherwise provided with a port 110 through which cement may be dispensed. The push handle 106 may be bonded to the push tube 104. The inflation tube 102 may be bonded to the inflation luer 108. The push tube 104 may be configured to slide over the inflation tube 102 and the inflation luer 108 may be configured to be received by the push handle 106.

FIG. 2b illustrates the delivery catheter 100 of FIG. 2a with a balloon 120 provided over a distal end of the delivery catheter 100 for insertion into the target area, such as a vertebral body. Prior to delivery of the encapsulation balloon 120, a balloon tamp may be inserted into the vertebral body and inflated to elevate a fracture in the vertebra. Alternatively, the encapsulation balloon may be used to elevate the fracture. The delivery catheter 100 may be used to deliver the encapsulation balloon 120 to the vertebral body and position the encapsulation balloon in the elevated fracture (or within the vertebral body to elevate the fracture). The delivery catheter 100 may further be used to inject cement into the encapsulation balloon to maintain the fracture in an elevated position. In the embodiment shown in FIGS. 2a and 2b, cement is injected into the encapsulation balloon via a side port 110.

In one embodiment, the balloon tamp for inflation and initial elevation is a a kyphoplasty balloon, and is separate from the encapsulation balloon. In another embodiment, the encapsulation balloon is used to elevate the fracture and no separate balloon tamp or kyphoplasty balloon is provided.

In general, it may be desirable inject 1 ml-6 ml of cement into the encapsulation balloon to approximately fill the created cavity or vertebral space. In one embodiment, the balloon receives approximately 2 ml of cement. The size of the encapsulation balloon and the volume of cement injected into the encapsulation balloon may vary based on the size of the vertebral body being treated. In some embodiments, more than one encapsulation balloon may be used to repair the vertebral body with the plurality of balloons being provided side by side, one in front of the other, and/or stacked. In one embodiment, the encapsulation balloon has an inner lumen diameter of 1.5 mm and an outer diameter of 2 mm.

In some embodiments, the encapsulation balloon may be provided with a radiopaque marker such that it may be viewed under fluoroscopy to ensure proper positioning before being injected with cement. Injection of the encapsulation balloon with cement may be done via a distal port on the catheter or a side port on the catheter. A distal port may have a diameter substantially equal to the diameter of the catheter.

Accordingly, in one embodiment, a delivery catheter including a central tube, also referred to as an inflation tube, having a delivery cannula and a distal end including an injection port is provided. An encapsulation balloon is provided over the distal end of the delivery cannula and in communication with the injection port. The delivery catheter is used to place the encapsulation balloon in a vertebral body and may be inserted through an access catheter. A radiopaque marker may be provided on the encapsulation balloon and/or on a distal portion of the delivery catheter to ensure correct placement of the encapsulation balloon. Cement may be injected through the delivery cannula, out of the port, and into the encapsulation balloon to inflate the encapsulation balloon. The encapsulation balloon may be uncoupled from the delivery catheter and the delivery catheter removed. The encapsulation balloon then remains in the vertebral body and the cement hardens. In some embodiments, the encapsulation balloon biodegrades or bioresorbs after the cement hardens.

In some embodiments, air may be removed from the delivery catheter (also referred to as a delivery cannula) before cement is injected through the delivery catheter into the encapsulation balloon. Referring back to FIG. 1b, air may be removed and a 3-way valve 204 used to maintain a vacuum. More specifically, air may be pulled into a VacLock syringe 206. The 3-way valve 204 may then be manipulated such that the delivery catheter 200 is open only to a syringe in contact with the cement reservoir. It is to be appreciated that a 3-way valve 204 such as shown in FIG. 1b may be interposed in the system embodiment shown in FIGS. 2a and 2b.

In some embodiments, a one-way valve may be provided as part of the delivery catheter or as part of the encapsulation balloon to prevent cement from escaping the encapsulation balloon. More specifically, the cement may be injected through the one-way valve to inflate the balloon with the one-way valve preventing cement from escaping the balloon.

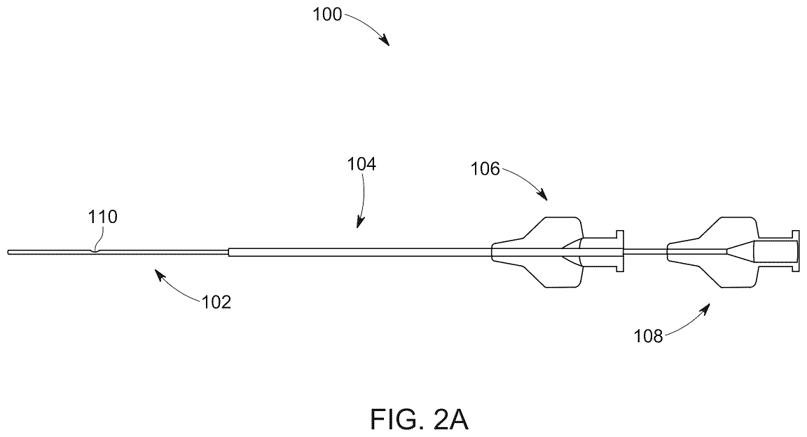

FIG. 3a illustrates an access catheter 8 for accessing a vertebral body. A tool such as a stylet may be provided central to the access catheter to assist in positioning the access catheter 8 to access the vertebral body. In other embodiments, the access catheter 8 may be provided with a trocar tip for accessing the vertebral body without a stylet. If a stylet is used, the stylet may be removed after access is made.

FIG. 3b illustrates a delivery catheter 10, in accordance with one embodiment. The delivery catheter 10 includes a central tube 20, also referred to as an inflation tube, having a distal end 13 and a proximal end 11. The proximal end may be considered the end of the catheter that is closest to the surgeon. The distal end may be considered the end of the catheter or balloon that is inserted into the patient and is furthest from the surgeon. A distal tip assembly 25 (also referred to as a catheter distal tip assembly) is provided at the distal end 13 and includes a port through which bone cement may exit the delivery catheter. An encapsulation balloon, also referred to as a balloon catheter, is provided at the distal tip assembly. A handle 16 may be removably coupled to the central tube 20.

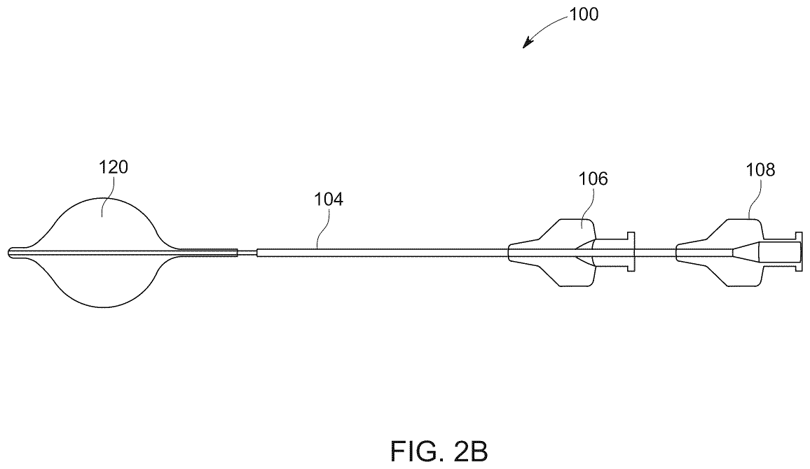

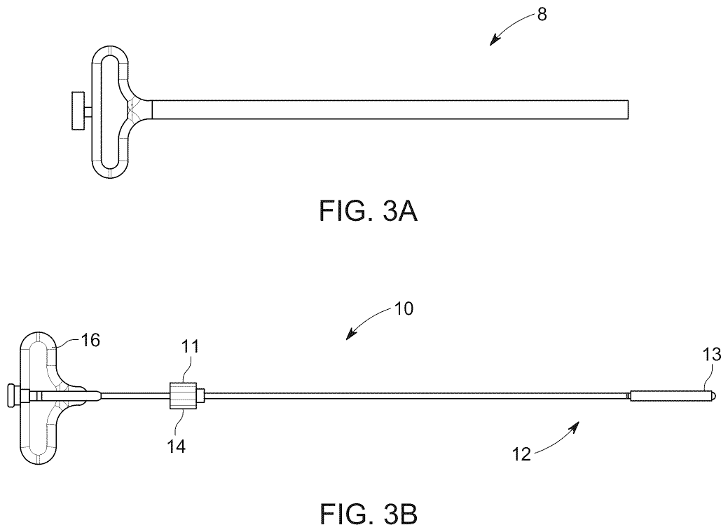

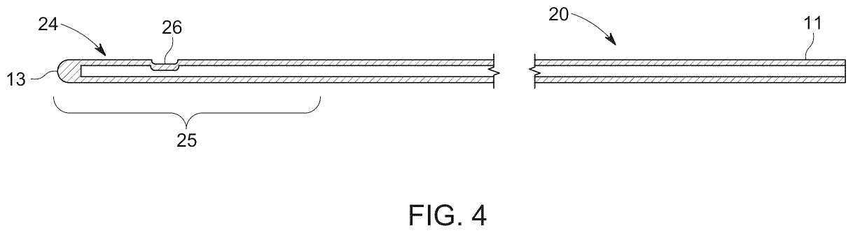

FIGS. 4, 5a, and 5b illustrate close up views of aspects of a distal portion of the delivery catheter, in accordance with one embodiment. FIGS. 6, 7, and 8 illustrate close up cross sectional views a distal portion of the delivery catheter with a distal tip assembly provided thereon, in accordance with one embodiment. The delivery catheter includes a central tube 20, also referred to as an inflation tube, comprising a delivery cannula. This may be, for example, a laser cut hypotube 27. A distal tip assembly 25 is provided at the distal end 13. The distal tip assembly 25 may comprise a plurality of layers. A biodegradable substrate or coating 24 is provided over the central tube. A silicone tube 22 is provided over at least a portion of the biodegradable substrate. A portion of the central tube 20, at or near the distal tip assembly, may be skived to provide an opening 26, or injection port, through which cement may be directed from the central tube 20 to an encapsulation balloon 21. The silicone tube 22 can act as a one-way valve over the opening 26. In the embodiment shown, the opening 26 is a side port. In other embodiments, an end port may be provided at the end of the central tube 20 through which the cement may be injected into the encapsulation balloon.

The encapsulation balloon 21 may be provided over the silicone tube 22 and going around the distal tip of the central tube 20. In some embodiments, the silicone tube 22 may act as a one-way inflation valve for the balloon 21. In other embodiments, the silicone tube 22 may be omitted.

A deployment tube 30, such as a PTFE tube, may be provided over the encapsulation balloon 21. This deployment tube 30 assists to maintain integrity of the encapsulation balloon 21 during deployment through the central tube 20.

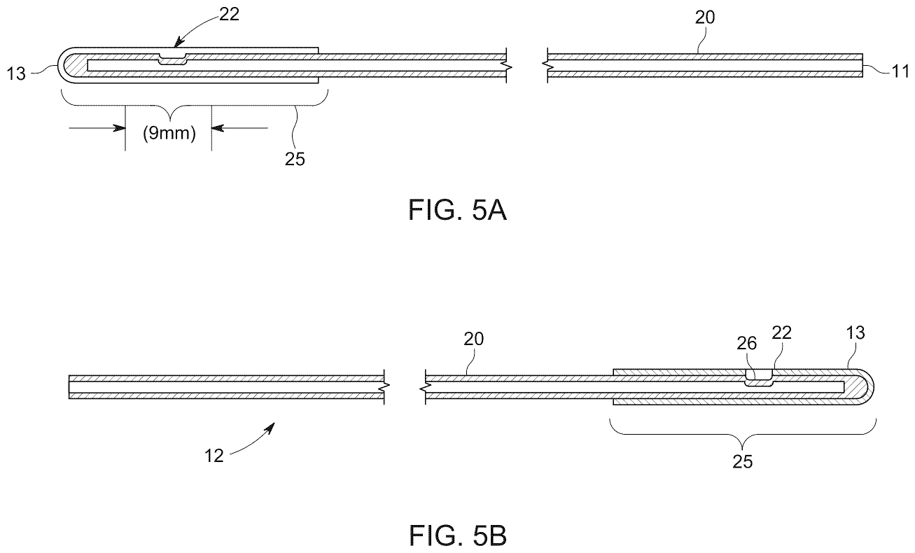

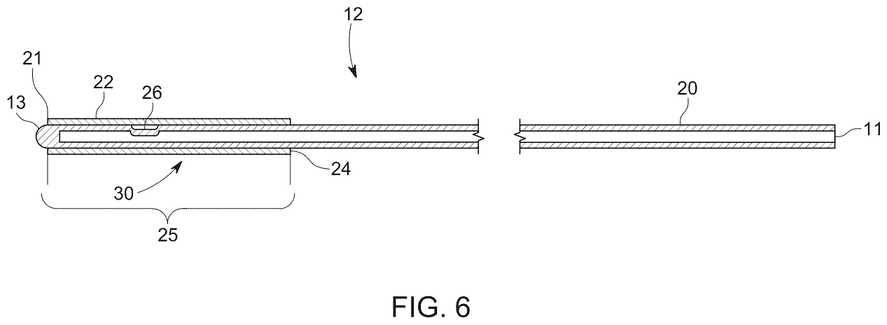

A connector piece 14 may be provided along the central tube 20. A dispensing tip 32 may be provided at a proximal end of the central tube 20. The dispensing tip 32 may be configured to receive a cement cannula. A radiopaque marker may be provided over the central tube 20. The radiopaque marker may be, for example, a marker band. In the embodiment shown in FIGS. 7 and 8, the marker band 32 extends from the connector piece 14 to a position just proximal of the distal tip assembly 25. In some embodiments, a radiopaque marker may be provided on the encapsulation balloon 22.

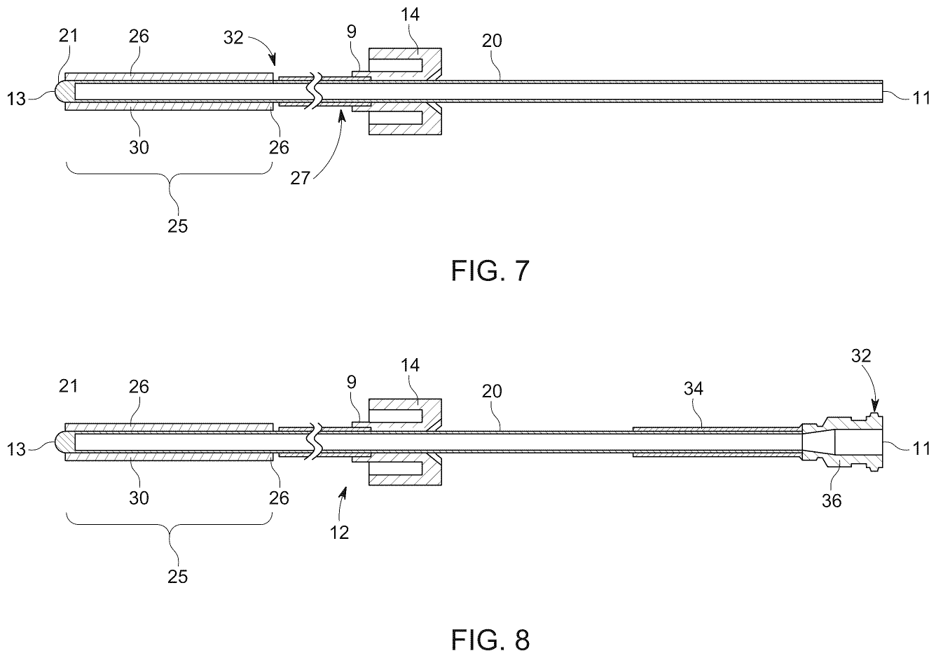

FIGS. 9a and 9b illustrate further aspects of the connector piece 14. In one embodiment, the connector piece 14 is a male luer connector. As shown, the connector piece 14 includes a recess with stops 19 for receiving the central tube 20 as encircled by marker band 32. An adhesive may be used to adhere the connector piece 14 and the portion of the central tube it surrounds. The connector piece 14 may substantially prevent cement filling devices from detaching from the central tube 20.

Returning to FIG. 8, a dispensing tip 32 (also referred to as a dispenser tip or inflation luer) may be provided at the proximal end 11. The dispensing tip may comprise a dispensing tip body 36 and an extension 34. An extension 34 may at least partially encircle the central tube 20. The extension 34 may be bonded, for example by soldering, to the central tube 20 and the body 36 of the dispensing tip at proximal and distal ends thereof. In one embodiment, the dispensing tip 32 is a 12 GA SS dispensing tip. The dispensing tip body 36 may receive an end of a cement cannula such that cement is injected through the dispensing tip and into the central tube 20.

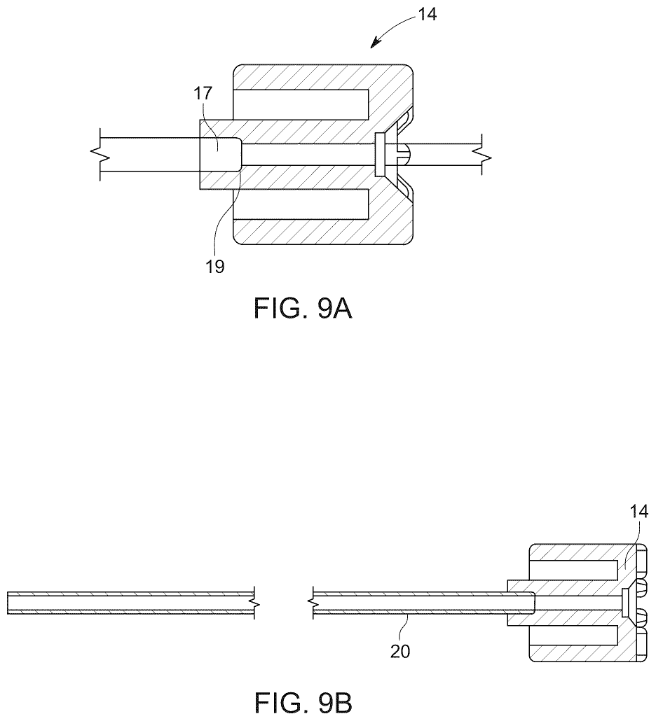

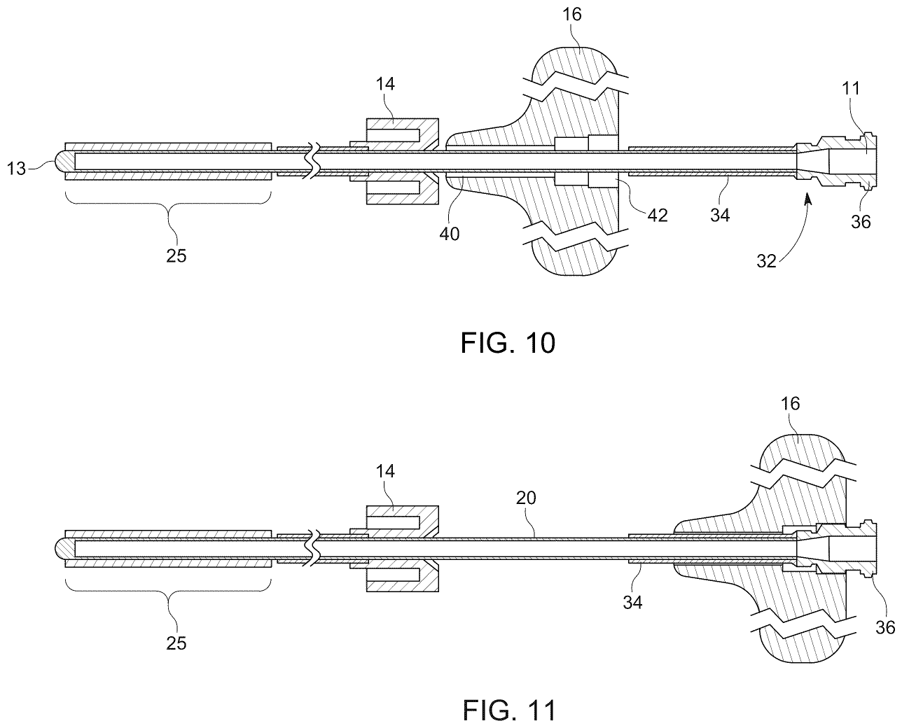

FIGS. 10 and 11 illustrate a handle 16 (also referred to as a push handle) for the delivery catheter, the handle 16 being provided over the central tube 20, in accordance with one embodiment. The handle 16 includes an internal slot 40 and may comprise two interlocking parts. The handle 16 may be assembled over the central tube 20 with the central tube 20 extending through the internal slot 40. The handle 16 may be assembled over the tube 20 distal of the dispensing tip extension 34. As assembled, the handle may have a recess 42 generally complementary in shape to the dispensing tip body 36. After assembly over the central tube 20, the handle 16 may be pushed proximally until the recess 42 bottoms against the dispensing tip body 36. This may result in a snap fit connection between the handle 16 and the dispensing tip body 36.

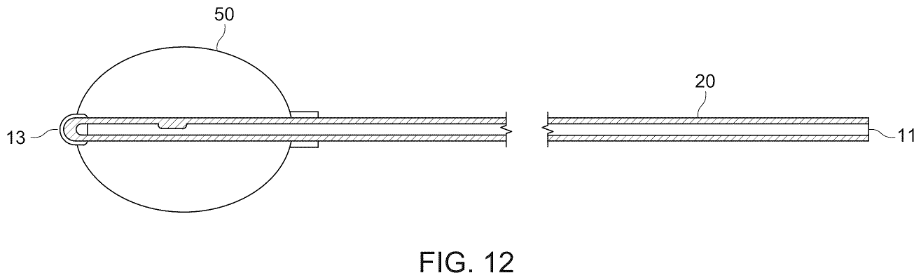

FIG. 12 illustrates the delivery catheter with a biodegradable, or bioresorbable, balloon 22 inflated at the distal end 13.

As previously discussed, in some embodiments, it may be desirable to remove air from the delivery catheter and the balloon prior to injection of cement into the balloon. This may be done by, for example, by suction of the air, pushing of the air, or evacuation of the air.

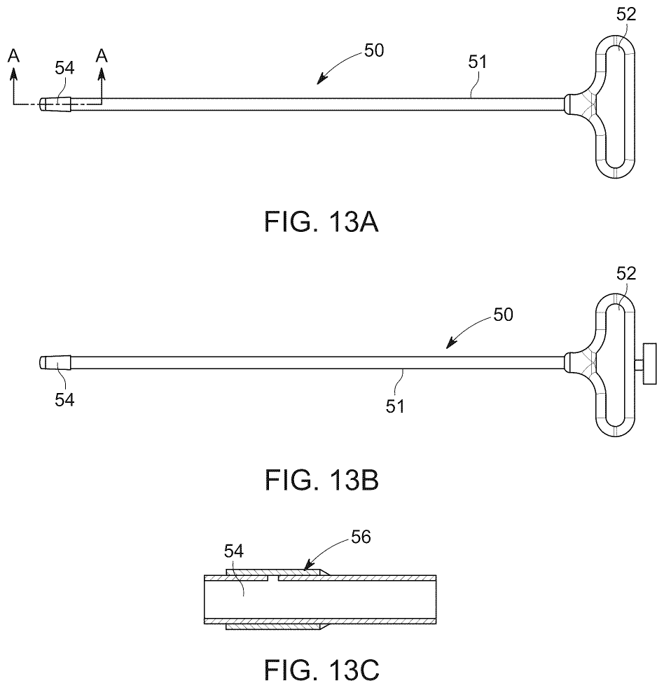

FIGS. 13a, 13b, and 13c illustrate a cement cannula 50, in accordance with one embodiment. The cement cannula 50 is used with the delivery catheter to inject cement from the cement cannula, through the central tube (also referred to as the inflation tube), and into the balloon. The cement cannula 50 includes a central cannula 51, a handle 52, and distal tip 54 (also referred to as a reservoir distal tip). The distal tip 54 may include a luer slip 56 and may be configured to engage with the luer lock 14 or the dispensing tip 32. FIGS. 13a and 13b illustrate the full cement cannula 50. FIG. 13c illustrates a distal tip 54 of the cement cannula 50.

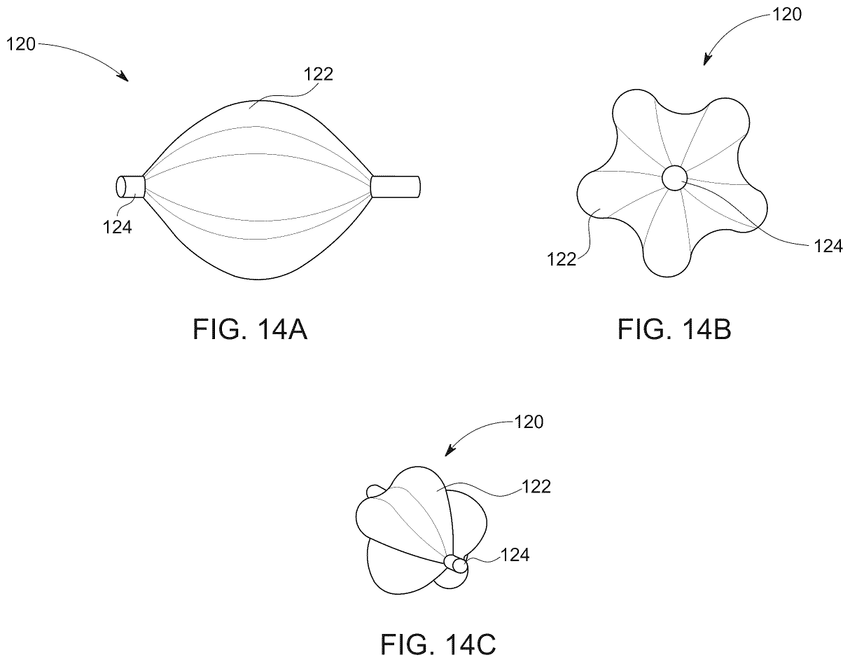

FIGS. 14a, 14b, and 14c illustrate an encapsulation balloon, in accordance with one embodiment. The balloon is adapted to have a relatively compact non-inflated configuration during deployment and to expand when filled with cement. The balloon includes an exterior inflatable portion and an interior shaft. The interior shaft may include a port to align with the port of the central tube (or inflation tube) of the delivery system. As shown, the exterior inflatable portion of the encapsulation balloon 120 may have a ridged or star shape and a distal tip 124. The ridged shape may be formed by providing a plurality, such as five, ridges 122 or wings. In a non-inflated configuration, the ridged shape folds and has a reduced crossing profile. In the inflated configuration, the ridged shape provides increased surface area and may enhance integration of the cement inflated balloon with surrounding bone (and, optionally, cement).

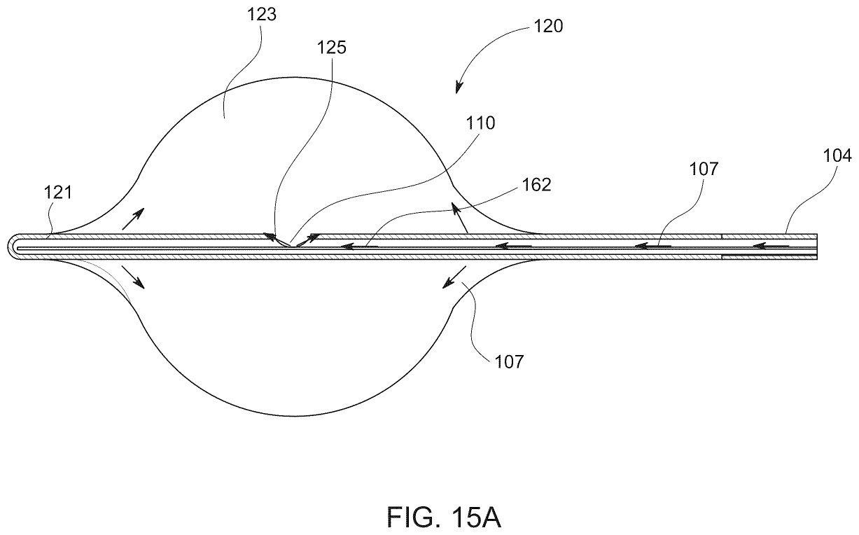

FIG. 15a illustrates inflation of the encapsulation balloon 120. As shown, the encapsulation balloon 120 comprises an exterior inflatable portion 123 and an interior shaft 121. For inflation, port 125 of the interior shaft 121 is aligned with port 110 of the central tube 102. FIG. 15b illustrates deployment of the encapsulation balloon 120. As shown in FIG. 15a, cement (shown by arrows 107 is pushed through the inflation tube 102 and out of the opening 110. As previously discussed, a silicone tube can be provided over the opening 110 and acting as a one-way valve. As a result, pressure in the balloon does not drop during inflation. As shown in FIG. 15b, after the balloon 120 has been inflated, the push tube 104 is pushed against the balloon and the delivery system is removed.

Detail will now be given of preparing a cement encapsulation catheter for use and using a cement encapsulation catheter for a vertebral augmentation procedure such as vertebroplasty or kyphoplasty.

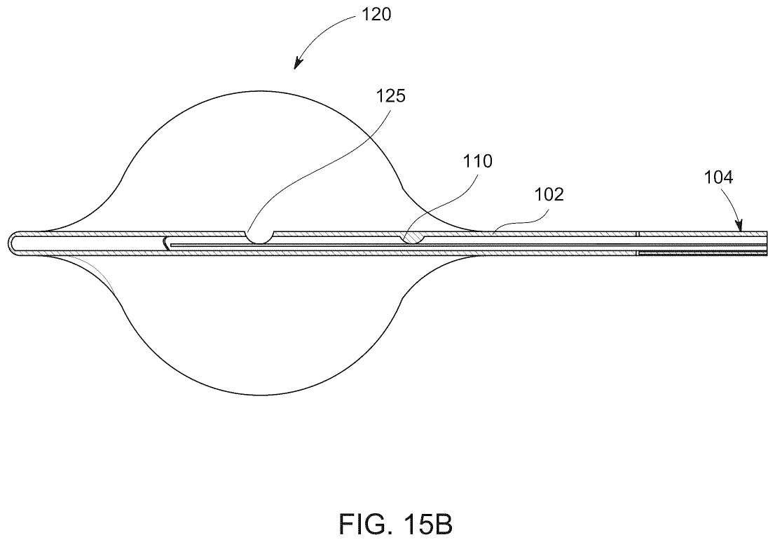

FIGS. 16a, 16b, and 16c illustrate the steps of preparing of a cement encapsulation catheter (also referred to as a delivery catheter) for use, in accordance with one embodiment. The cement encapsulation catheter is generally provided in a sterile packaging. FIG. 16a illustrates removal of the cement encapsulation catheter 220 from the sterile packaging. As packaged, the cement encapsulation catheter 220 may include a balloon protective sheath 222 over the encapsulation balloon at the distal end of the cement encapsulation catheter 220. Before use, the balloon protective sheath is removed, exposing the encapsulation balloon 224, shown in FIG. 16b. As shown in FIG. 16c, a stiffening wire handle 226 may be provided inserted in the cement encapsulation catheter 220. The stiffening wire may be removed after insertion of the distal end of the balloon catheter 220 into the vertebra body.

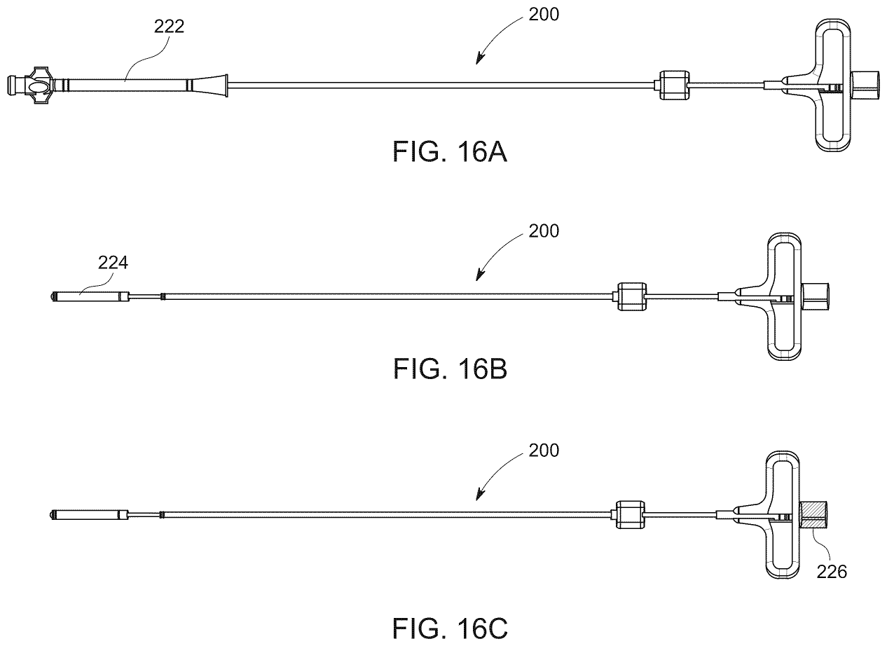

The cement reservoir 212 may have an injection port 214, a proximal end 216, a cap (not shown) at the proximal end 216, and a distal end having a distal tip 218 for coupling, directly or indirectly, with the cement encapsulation catheter. Cement is received by the cement reservoir 212 from the cement source 230 such as a cement mixer. In some embodiments, the cement may be mixed in a bone cement mixer and the bone cement mixer 230 coupled to the cement reservoir 212 to fill the cement reservoir with bone cement. FIG. 17 illustrates such coupling and filling. With reference to FIG. 1b, the flexible tube 208 is fitted to the cap of the cement reservoir 212 and the distal tip 208 of the cement reservoir 212 is coupled to the 3-way valve or stopcock 204 for indirect coupling to the cement encapsulation catheter or delivery catheter 200.

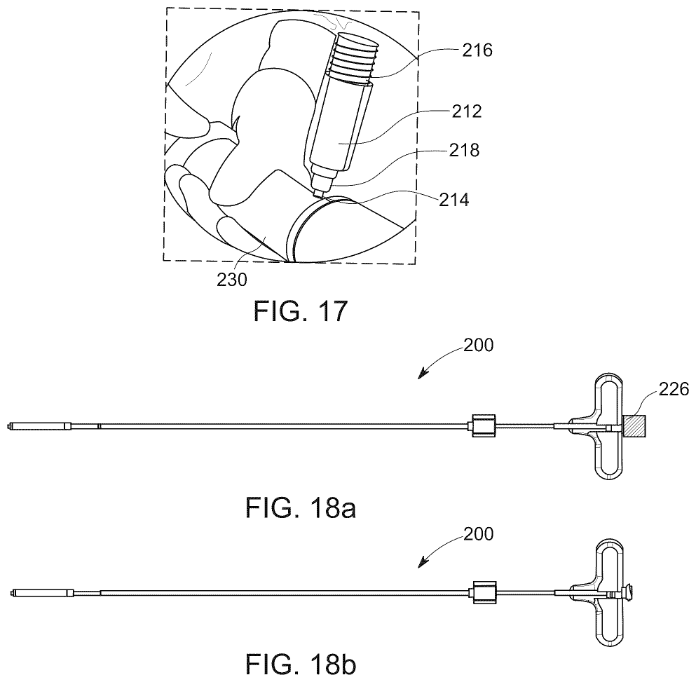

As discussed, the delivery catheter 200 may be provided with a stylet (or stiffening wire) 226 inserted therein. Prior to connection of the 3-way stopcock or valve 204 (shown in FIG. 1b) to the cement encapsulation catheter 200, the stylet (or stiffening wire) 206 is removed. FIG. 18a illustrates the stylet 226 in place. FIG. 18b illustrates the stylet 226 removed. After removal, the 3-way stopcock may then be coupled to the cement encapsulation catheter. This may be done via a threaded connection, in one embodiment. A VacLok syringe my then be attached to the 3-way stopcock. The 3-way stopcock or valve has 3 connection points. At this stage, the reservoir is attached to a first connection point, the cement encapsulation catheter is attached to a second connection point (for example, opposite the first connection point), and the VacLok syringe is attached to the third connection point (for example, between and at a 90° angle to the first and second connection points). The 3-way stopcock is adjusted to position a valve to completely block the cement reservoir.

Vacuum is applied inside the cement encapsulation catheter using the VacLok syringe. While maintaining vacuum in the VacLok syringe, the 3-way stopcock is manipulated such that a valve completely blocks the VacLok syringe. This allows cement to flow from the cement reservoir into the cement encapsulation catheter. The pump handle can be rotated to introduce the cement into the cement encapsulation catheter. Fluoroscopic imaging may be used throughout the procedure to verify and monitor cement flow as appropriate. At any point during introduction of the cement, cement flow may be stopped by rotating the pump handle counter-clockwise until force-free handle rotation is achieved. When an appropriate amount of cement has been introduced, cement introduction may be stopped (such as by rotating the pump handle counter-clockwise), and the cement reservoir may be removed.

Once filled with cement, the encapsulation balloon is removed from the delivery system and the delivery system is removed. Removing the encapsulation balloon from the delivery system may comprise pushing the encapsulation balloon from the cement encapsulation catheter. This may be done by using a push tube such as described in FIG. 2a. The delivery system may be removed using rotational oscillating motions.

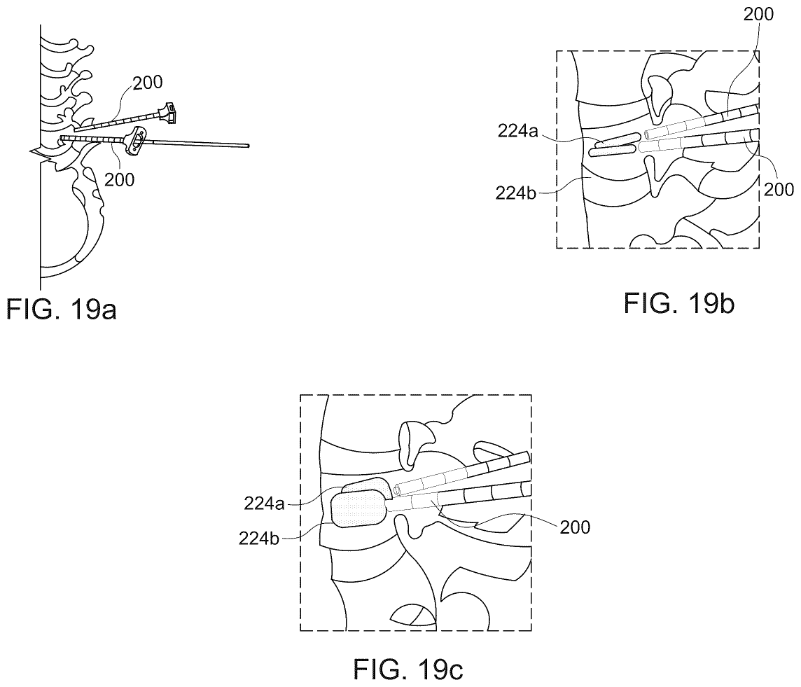

In some embodiments, two cement encapsulation balloons may be inserted into a vertebral body. FIGS. 19a, 19b, and 19c illustrate deploying two cement encapsulation balloons 224 into a vertebral body. FIG. 19a illustrates introduction of one or more delivery catheters 200 to the vertebral space. FIG. 19b illustrates deployment of cement encapsulation balloons 224a and 224b through the delivery catheter(s) 200. FIG. 19c illustrates inflation of the cement encapsulation balloons 224a and 224b. As shown, the delivery catheter 200 may be inserted with an attached cement encapsulation balloon 226. This may be done under lateral x-ray control. The position of the balloon may be checked under x-ray control and confirmed. In general, it is desirable that the whole balloon portion, including the shaft, is positioned completely inside the vertebra with the balloon catheter exiting the delivery catheter. This can be repeated on the contralateral side. Cement is injected through the cement encapsulation catheter 200 and into the encapsulation balloon 224, 224a, and/or 224b. The volume of cement used can be tracked using volume indicators on the cement reservoir(s).

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A system for performing a vertebral augmentation procedure, the system comprising:
a delivery device, the delivery device comprising:
an inflation tube having a delivery cannula and an injection port, wherein bone cement can flow through the delivery cannula and out of the injection port;
a push tube provided over a proximal portion of the inflation tube; and
a push handle; and
an encapsulation balloon configured for receiving bone cement, the encapsulation balloon being provided over a distal portion of the inflation tube such that the port is provided within the encapsulation balloon.

2. The system of claim 1, further comprising an access catheter.

3. The system of claim 1, further comprising a balloon tamp.

4. The system of claim 1, wherein the delivery device further comprises an inflation luer for receipt by the push handle.

5. The system of claim 1, wherein the port is a side port.

6. The system of claim 1, wherein the encapsulation balloon is biodegradable and/or bioresorbable.

7. The system of claim 1, wherein the encapsulation balloon includes a radiopaque marker.

8. The system of claim 1, wherein the encapsulation balloon includes an exterior inflatable portion and an interior shaft, wherein the interior shaft includes a port configured to align with the injection port.

9. The system of claim 8, wherein the exterior inflatable portion of the encapsulation balloon comprises a ridged shape having a distal tip.

10. The system of claim 8, wherein the port includes a one-way valve.

11. A system for performing a vertebral augmentation procedure, the system comprising:
a delivery catheter, the delivery catheter comprising:
a central tube having a distal end and a proximal end;
a distal tip assembly provided at the distal end, the distal tip assembly including a port, wherein bone cement can flow through the central tube, to the distal tip assembly, and out of the port;
a dispensing tip, wherein the dispensing tip is configured for receiving a cement cannula; and
a handle removably coupled to the central tube and configured to receive the dispensing tip; and
an encapsulation balloon configured for receiving bone cement, wherein the encapsulation balloon is provided over the distal tip assembly.

12. The system of claim 11, wherein the handle comprises an internal slot and two interlocking parts.

13. The system of claim 11, further comprising a cement cannula, the cement cannula comprising a central cannula, a handle, and a distal tip, wherein the distal tip engages with the dispensing tip of the delivery catheter.

14. The system of claim 11, wherein the encapsulation balloon is biodegradable and/or bioresorbable.

15. The system of claim 11, wherein the encapsulation balloon includes a radiopaque marker.

16. The system of claim 1, wherein the encapsulation balloon includes an exterior inflatable portion and an interior shaft, wherein the interior shaft includes a port configured to align with the injection port.

17. The system of claim 16, wherein the exterior inflatable portion of the encapsulation balloon comprises a ridged shape having a distal tip.

18. The system of claim 16, wherein the port includes a one-way valve.

19. A system for performing a vertebral augmentation procedure, the system comprising:
a cement encapsulation system comprising:
a cement reservoir having a proximal end and a reservoir distal tip;
a stopcock;
a syringe for removing air; and
a tube;
a catheter delivery system comprising:
a delivery catheter, the delivery catheter comprising:
a central tube having a distal end and a proximal end;
a catheter distal tip assembly provided at the distal end, the distal tip assembly including a port, wherein bone cement can flow through the central tube, to the distal tip assembly, and out of the port; and
a handle removably coupled to the central tube;
wherein the cement reservoir is removably coupled to the delivery catheter; and
an encapsulation balloon configured for receiving bone cement, wherein the encapsulation balloon is provided over the catheter distal tip assembly.

20. The system of claim 19, wherein the cement reservoir is removably coupled to the delivery catheter via the stopcock.
